(19) United States
(12) Plant Patent Application Publication
(10) Pub. No.: US 2007/0214523 P1
(43) Pub. Date: Sep. 13, 2007

(54) Peach tree named 'NJ350'

(75) Inventors: Joseph C. Goffreda, Manalapan, NJ (US); Anna M. Voordeckers, East Windsor, NJ (US)

Correspondence Address: James A. Lucas;Driggs, Hogg & Fry CO., L.P.A., 38500 Chardon Road, Willoughby Hills, OH, 44094, US

(73) Assignee: Rutgers, The State University, New Brunswick, NJ (US)

(21) Appl. No.: 11/371,384

(22) Filed: Mar. 9, 2006

(51) Int. Cl. A01H 5/00

(52) U.S. Cl. PLT/194

(57) ABSTRACT

A new and distinct peach variety of Prunus persica named ‘NJ350’ is provided. This variety is distinguished from other peach varieties by its unique combination of nonshowy flowers, fruit that ripen early in the season, attractive fruit with an orange-red ground color, clingstone fruit with a juicy, melting texture and moderately acidic flavor, and good production of firm fruit with a low incidence of split pits for the season.

### CROSS REFERENCE TO RELATED APLICATIONS

NONE

### STATEMENT REGARDING FEDERALLY SPONSORED RESEARCH OR DEVELOPMENT

NONE

### LATIN NAME OF GENUS AND SPECIES OF THE PLANT CLAIMED

Prunus persica L.

### CULTIVAR NAME

NJ350

### BACKGROUND OF THE INVENTION

The present invention relates to a new and distinct variety of peach tree named ‘NJ350’. Our new tree resulted from crossing ‘Garnet Beauty’ (unpatented) as the seed parent with ‘Sentry’ (unpatented) peach tree, as the pollen parent. The new variety differs from seed parent ‘Garnet Beauty’ in that the new variety has fruit with a rounded apex having an inconspicuous apex point, while the seed parent often has fruit with a prominent apex point. The new variety differs from pollen parent ‘Sentry’ in that the new variety has reniform leaf glands, while the parent has globose leaf glands. The new variety also ripens 7 to 10 days earlier than comparably cropped trees of either the seed or pollen parents. The resulting tree was selected when growing in a cultivated area as the 152^{nd} tree in the 81^{st} row of Block D at the Rutgers Fruit Research and Extension Center in Cream Ridge, N.J.

### BRIEF SUMMARY OF THE INVENTION

The ‘NJ350’ variety is distinguished from other peach varieties due to the following unique combination of characteristics:
Attractive round fruit with an inconspicuous apex point.
Fruit with an orange-red ground color.
Low incidence of split pits for season.
Good production of firm fruit.

The variety was asexually reproduced at the Rutgers Fruit Research and Extension Center in Cream Ridge, N.J. Asexual reproduction of this new variety by budding onto ‘Lovell’ rootstock (unpatented) shows that the foregoing characteristics are so reproduced.

The following detailed description concerns the original tree, ‘NJ350’. The original tree and asexual progeny have been observed growing in a cultivated area at the Rutgers Fruit Research and Extension Center in Cream Ridge, N.J. Certain characteristics of this variety, such as growth and color, may change with changing environmental conditions (such as, light, temperature, moisture, nutrient availability) or other factors. Color descriptions and other terminology are used in accordance with their ordinary dictionary descriptions, unless the context clearly indicates otherwise. Color designations are made with reference to The Royal Horticultural Society (R.H.S.) Colour Chart.

### BRIEF DESCRIPTION OF THE DRAWINGS

This new variety is illustrated by the accompanying photographic drawings, depicting the peach tree by the best possible color representation using color photography. Colors are approximate as color depends on horticultural practices, such as light level, fertilization rate, and other conditions and, therefore, the color characteristics of this new variety should be determined with reference to the observations described herein, rather than from these illustrations alone.

FIG. 1 is a color photograph taken on Aug. 29, 2005 of a characteristic twig of ‘NJ350’ in late summer bearing typical leaves of the mature foliage.

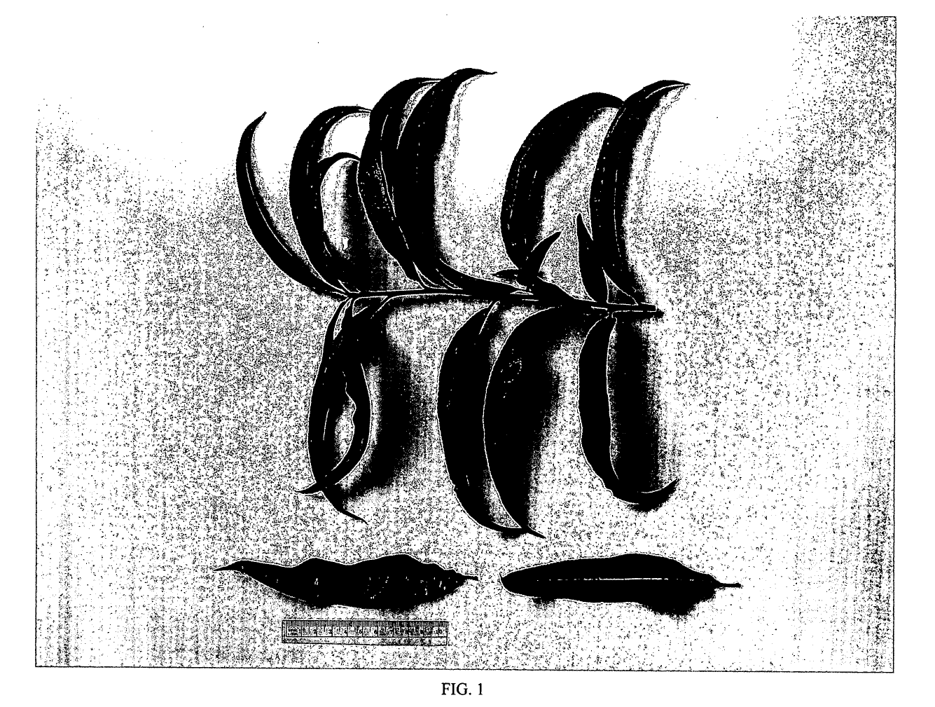

FIG. 2 is a color photograph of mature fruit of ‘NJ350’ and stones harvested from the Rutgers Fruit Research and Extension Center in Cream Ridge, N.J. on Jul. 25, 2005. Whole fruit are presented in three positions and a transverse cross section to show that the pericarp adheres to the pit when the fruit is mature. The stones illustrate the medium, narrow, and fine lines of the dorsal ridge and the pit grooves on the surface of the stone.

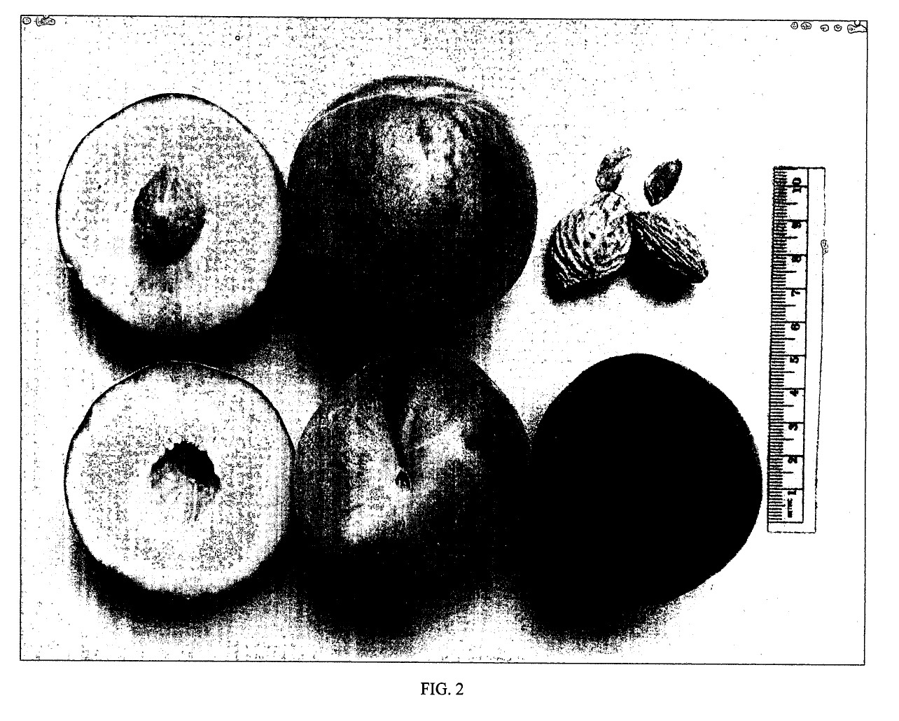

FIG. 3 is a color photograph of a characteristic twig that illustrates the typical flower buds and small, nonshowy flowers of ‘NJ350’ observed on a tree at the Rutgers Fruit Research and Extension Center in Cream Ridge, N.J. on Apr. 21, 2004.

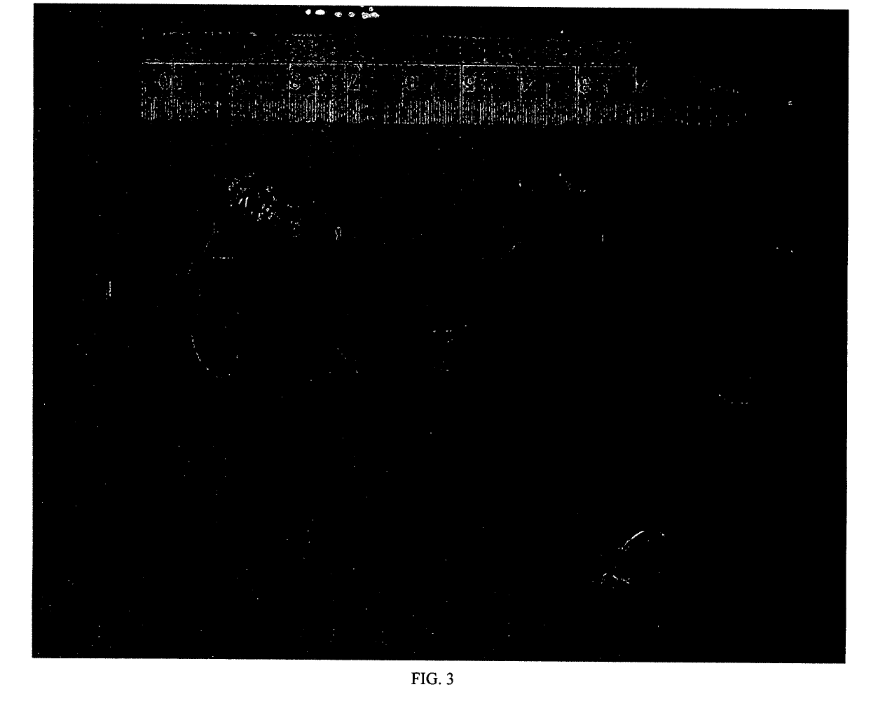

FIG. 4 is a color photograph of a tree of ‘NJ350’ in early fall that illustrates the spreading growth habit of a tree at the Rutgers Fruit Research and Extension Center in Cream Ridge, N.J. on Oct. 27, 2005.

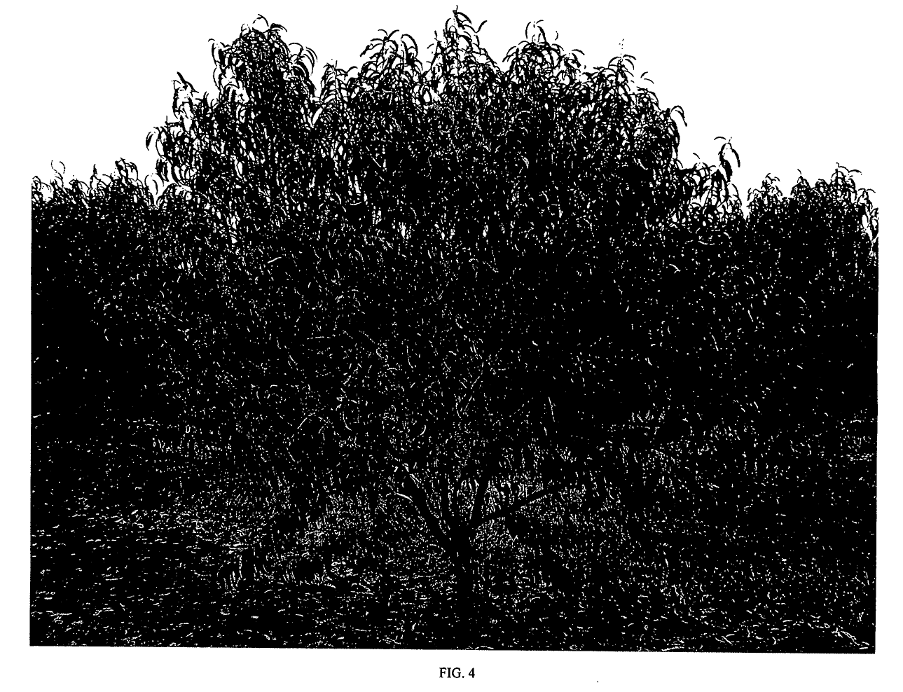

FIG. 5 is a color photograph taken on Oct. 27, 2005 of immature bark of ‘NJ350’ that illustrates color and the moderate density of elliptical lenticels on the immature bark.

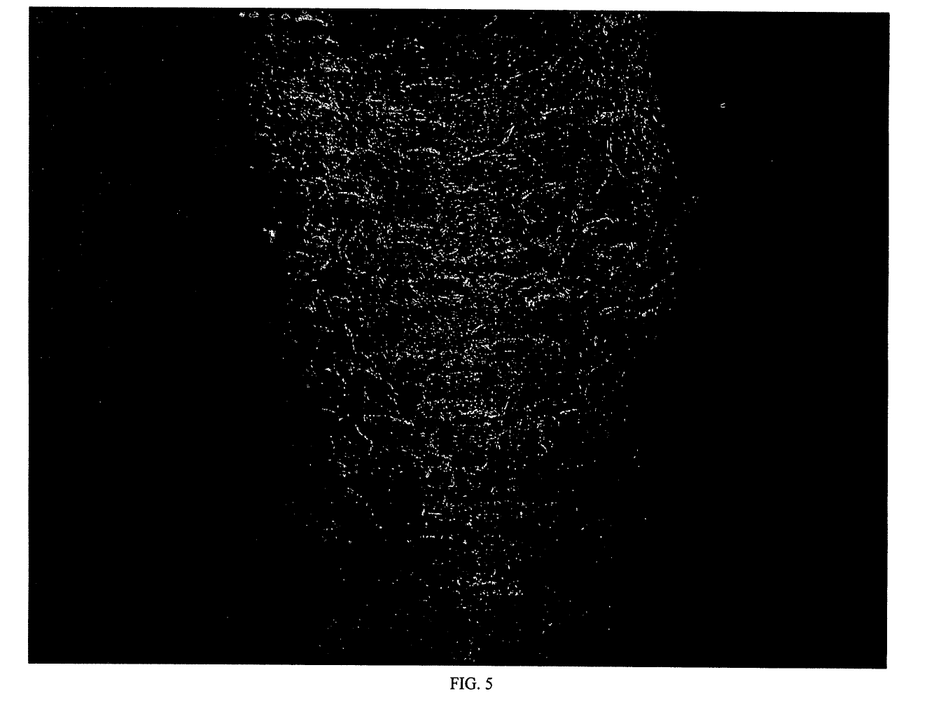

FIG. 6 is a color photograph taken on Oct. 27, 2005 of mature bark of ‘NJ350’ that illustrates the fairly smooth texture and prominent lenticels of the mature bark.

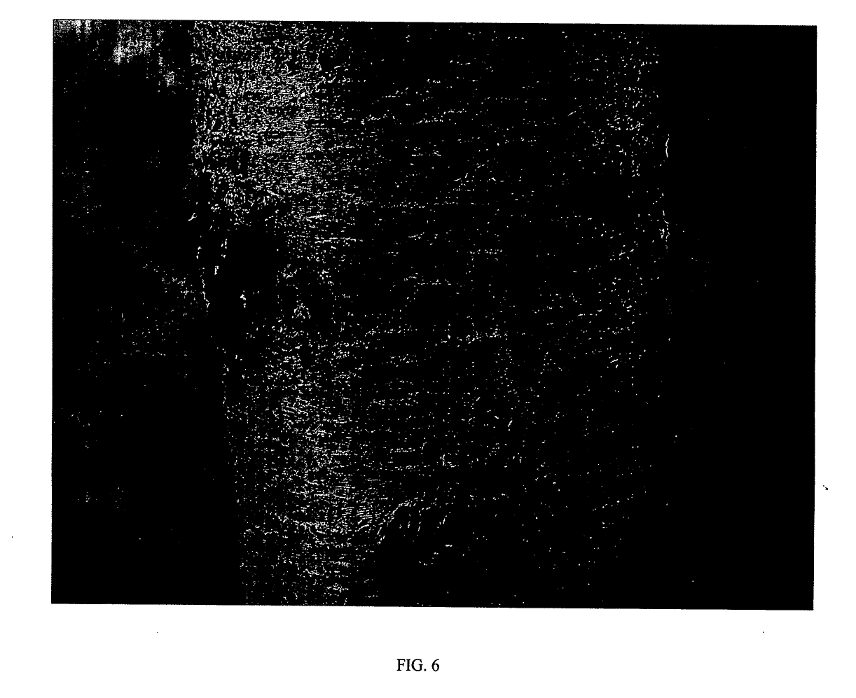

### DETAILED BOTANICAL DESCRIPTION

The following detailed description of the ‘NJ350’ variety is based on observations of an asexually reproduced tree. The observed tree was four years of age and growing on ‘Lovell’ seedling rootstock (unpatented) in Research Block E at the Rutgers Fruit Research and Extension Center in Cream Ridge, N.J.
Scientific name: Prunus persica L.

Parentage:

| | |
|---|---|
| Seed parent: | Garnet Beauty. |
| Pollen parent: | Sentry. |

Tree:

| | |
|---|---|
| Vigor: | Vigorous. |
| Plant hardiness zone: | Growth of plants has only been observed in zone |
| | 6b. |
| Dormant flower bud | At least to −16° C. |
| cold tolerance: | |
| Overall shape: | Spreading. |
| Height: | Above average as compared to other peach |
| | cultivars. For example, measurement of a |
| | typical grafted tree on ‘Lovell’ seedling root- |
| | stock (unpatented) at four years after planting |
| | shows an average height of 3.4 meters when |
| | grown in Cream Ridge, New Jersey. |
| Width: | Average as compared to other peach cultivars. |
| | For example, measurement of a typical grafted |
| | tree on ‘Lovell’ seedling rootstock (unpatented) |
| | at four years after planting shows an average |
| | width of 4.6 meters when grown in Cream |
| | Ridge, New Jersey. |
| Caliper: | Four year old tree is 32 cm in circumference |
| | measured at 20 cm from the ground. |

Trunk and branches:

| | |
|---|---|
| Trunk bark | Fairly smooth with prominent lenticels becoming rough as |
| texture: | the tree ages. |
| Trunk bark | Under color is greyed-white (RHS 156c). |
| color: | |
| Primary | Branches that are approximately 15 cm in circumference |
| branches: | are greyed-orange (RHS 177a) in color, overlaid with |
| | greyed-white (RHS 156a). |
| Lenticels: | Moderate density, approximately 2.5 per square cm; |
| | elliptical shape; typical examples of which measured 6 mm |
| | in length and 2.1 mm in width; greyed-orange (RHS 164a) |
| | in color. |
| Branch | None. |
| pubescence: | |
| New growth | Color between greyed-purple (RHS 183a) and greyed- |
| bark: | orange (RHS 166a) in sun; color yellow-green (between |
| | RHS 152a and RHS 152b) in shade. |
| Internodes: | Length averaging 28.8 mm on a one-year shoot. |

Leaves:

| | |
|---|---|
| Texture: | Glabrous. |
| Sheen: | Young leaves semi-glossy with a flat finish on the |
| | underside. |
| Length: | About 146 mm to 179 mm, averaging about 158 mm |
| | including the petiole. |
| Width: | About 31 mm to 38 mm, averaging about 34 mm. |
| Petiole: | Averaging 10.7 mm long and about 1.4 mm |
| | in diameter. |
| Margin: | Serrulate. |
| Margin undulation: | Nearly none to slight. |
| Form: | Elliptic. |
| Apex: | Sharply acute, curved downward. |
| Base: | Broadly acute. |
| Venation: | Pinnate. |
| Glands: | |
| Number: | About 2 to 5, averaging about 3.3. |
| Position: | Located primarily on the leaf margin. |
| Size: | Length averaging 1.0 mm and width |
| | averaging 0.6 mm. |
| Form: | Reniform. |
| Stipules: | None observed on mature leaves. |
| Leaf Color: | |
| Upper leaf surface: | Yellow-green (between RHS 146a and RHS 148a). |
| Lower leaf surface: | Yellow-green (RHS 147b). |
| Vein: | Greyed-yellow (RHS 160a). |
| Pubescence: | None. |

Flowers:

| Size: | Large size, typical flower measuring between 14.8 mm to |
|---|---|
| | 18.3 mm, averaging about 17.0 mm across. |
| Color: | |
| Dormant bud: | Greyed-green (between RHS 196b and RHS 198c). |
| Pink stage bud: | Red (between RHS 54b and RHS 54c). |
| Open flower: | Young open flowers red (between RHS 54c to RHS 54d), |
| | and venation becoming red-purple (RHS 66d) at |
| | petal fall. |
| Petals: | Typically five petals per flower; cupped and obovate in |
| | shape; averaging 11.9 mm long and 9.8 mm wide. Red |
| | (between RHS 54c and RHS 54d) in color. |
| Petal apex: | Rounded. |
| Petal base: | Cuneate. |
| Stamens: | |
| Number: | Variable, typical range 33 and 38, averaging 34.4. |
| Length: | Variable, between 11.5 mm to 14 mm, averaging |
| | 12.6 mm. |
| Filament color: | Green-white (RHS 157c). |
| Anther color: | Red (RHS 42a). |
| Pistil: | |
| Number: | One. |
| Size: | Length between 14.4 and 17.9 mm, averaging |
| | about 16.4 mm. |
| Pistil color: | Greyed-yellow (RHS 160a). |
| Ovary: | Dense pubescence and ellipsoid in shape. |
| Sepals: | |
| Number: | Five. |
| Pubescence: | Light. |
| Color: | Yellow-green (RHS 146c) with a greyed-red (RHS 182a) |
| | over color. |
| Shape: | Triangular, with a rounded apex. |
| Size: | Length averaging 4.8 mm, width averaging 3.9 mm. |
| Nectar cup | Greyed-orange (RHS 167a). |
| color: | |
| Pollen: | Abundant; yellow-orange (RHS 16a) in color. |
| Fragrance: | Very slight. |
| Bloom season: | Onset of bloom in 2005 on April 15; full bloom on April |
| | 18. |

Fruit:

| | |
|---|---|
| Size: | Large, averaging about 6.8 cm long, 6.7 cm |
| | wide parallel to the suture and 6.6 cm wide |
| | perpendicular to the suture. |
| Typical weight: | 162 g. |
| Form: | |
| Longitudinal section: | Round. |
| Traverse section: | Round. |
| Suture: | Very shallow, extending from the base to apex. |
| Ventral surface: | Nearly smooth. |
| Base: | Round. |
| Apex: | Round. |
| Stem: | Average length of 7.1 mm and an average |
| | diameter of 5.2 mm. |
| Skin: | |
| Thickness: | Average. |
| Surface: | Regular with short pubescence. |
| Tenacity: | Average. |
| Astringency: | None. |
| Tendency to crack: | Very low. |
| Color: | Mottled red (RHS 46a) over an orange-red |
| | (RHS 34b) blush; ground color orange-red |
| | (RHS 32d). |
| Fruit Properties: | |
| Flesh color: | Yellow-orange (RHS 15c). |
| Flesh adhesion: | Clingstone. |
| Juice: | Moderate. |
| Texture: | Firm, but melting. |
| Fibers: | Not noticeable. |
| Ripens: | Between June 27 and July 15 at Cream Ridge |
| | New Jersey. |
| Flavor: | Above average for season, moderately acidic. |
| Soluble solids: | 9.7%. |
| Aroma: | Very slight. |
| Eating quality: | Good. |
| Keeping quality: | Average. Has held its flavor and firmness |
| | for at least 7 days in cold storage at 1° C. to |
| | 4° C. |
| Shipping quality: | Very good. Fruit are generally very firm at |
| | harvest. No bruising or scaring disorders have |
| | been observed. |
| Usage: | Dessert. |
| Market: | Local and long distance. |
| Productivity: | Very good. Trees have produced a crop in 6 out |
| | of 8 years and a full crop in 6 out of 8 years |
| | at Cream Ridge, New Jersey. |

Stone:

| | |
|---|---|
| Type: | Clingstone. |
| Form: | Obovoid. |
| Base: | Medium. |
| Apex: | Medium. |
| Surface: | Pit grooves. |
| Ventral suture: | Medium. |
| Dorsal ridge: | Medium, narrow, and fine lines. |
| External color: | Orange-white (between RHS 159b and RHS 159c), with |
| | greyed-orange (between RHS 165b and RHS 165c) |
| | grooves. |
| Internal color | Orange-white (between RHS 159a and RHS 159b). |
| when cracked: | |
| Cavity | Greyed-orange (between RHS 165c and RHS 165d). |
| surface color: | |
| Average stone | 4.8 g. |
| dry weight: | |
| Average stone | Varies between 5.1 and 8.8 mm. |
| wall thickness: | |
| Size: | Averages about 36 mm long, 26 mm wide parallel to the |
| | dorsal ridge, and 20 mm wide perpendicular to the dorsal |
| | ridge. |
| Tendency to | Very low for ripening season. |
| split: | |
| Kernel: | |
| Form: | Irregular. |
| Skin color: | Greyed-orange (between RHS 165a and RHS 165b). |
| Vein color: | Greyed-orange (between RHS 165a and RHS 165b). |
| Viability: | No. |
| Size: | Highly variable; forms only rudimentary seed. |

Plant/fruit disease and pest resistance/susceptibility: No atypical resistances/susceptibilities have been noted under normal cultural practices.

We claim:

1. A new and distinct variety of peach tree, substantially as herein shown and described.
